(19) United States
(12) Patent Application Publication
(10) Pub. No.: US 2010/0092958 A1
(43) Pub. Date: Apr. 15, 2010

(54) Methods for Determining Collateral Artery Development in Coronary Artery Disease

(76) Inventors: Michael Simons, Hamden, CT (US); Thomas W. Chittenden, Boston, MA (US)

Correspondence Address: LICATA & TYRRELL P.C., 66 E. MAIN STREET, MARLTON, NJ, 08053, US

(21) Appl. No.: 12/444,423

(22) PCT Filed: Oct. 5, 2007
(86) PCT No.: PCT/US07/80506

§ 371 Date: Apr. 6, 2009

Related U.S. Application Data
(60) Provisional application No. 60/829,941, filed on Oct. 18, 2006.

(51) Int. Cl. C12Q 1/68; C12Q 1/00

(52) U.S. Cl. 435/6; 435/4

(57) ABSTRACT

The present invention relates to a method for determining collateral artery development in a human subject with coronary artery disease based upon the levels of expression of markers associated with collateral artery development in coronary artery disease.

### INTRODUCTION

This application claims benefit of U.S. provisional patent application Ser. No. 60/829,941 filed Oct. 18, 2006, the contents of which is incorporated by reference in its entirety.

This invention was made in the course of research sponsored by the National Institutes of Health (Grant No. HL53793). The U.S. government has certain rights in this invention.

### BACKGROUND OF THE INVENTION

Coronary artery disease (CAD) is the most common cause of morbidity and mortality in industrialized societies. Typically, the advancing atherosclerosis leads to narrowing or occlusion of major coronary arteries and their branches resulting in angina, heart failure or myocardial infarction. Clinical investigations suggest that a significant minority of CAD patients present with or develop in the course of their illness extra arterial conduits, termed coronary collaterals, which link proximal and distal parts of the arterial tree bypassing areas of stenosis and/or occlusion (Koerselman, et al. (2003) Circulation 107(19):2507-2511). Thus, collateral arteries function as “natural bypasses” effectively restoring the blood flow to compromised tissues. Moreover, the development of collateral circulation has been shown to play an important physiologic role in promoting survival and protecting tissues from ischemic damage (Hansen (1989) Am. Heart J. 117(2):290-295; Tayebjee, et al. (2004) QJM. 97(5):259-272), and its stimulation has emerged as one of the principal approaches to therapeutic angiogenesis (Simons and Ware (2003) Nat. Rev. Drug Discov. 2(11):863-871).

Collateral artery formation, also known as arteriogenesis, has been shown to be triggered by shear stress (Schaper, et al. (2003) Arterioscler. Thromb. Vasc. Biol. 23:1143-1151). Shear stress has been shown to upregulate ICAM-1 in cultured human saphenous vein endothelial cells (Sultan et al. (2004) FEBS Lett. 564(1-2):161-5) and induce the activity of Cdc42 and Rho (Li et al. (1999) J. Clin. Invest. 103(8):1141-50). Similarly, osteopontin is known to be upregulated during vascular remodeling and neointima formation in both rat models and human vascular diseases including atherosclerosis and restenosis (Giachelli et al. (1995) Ann. N.Y. Acad. Sci. 760:109-26). However, factors responsible for the presence or absence of collateral circulation have not been fully investigated. Certain predictors of collateral presence have been proposed including a history of angina (Fujita, et al. (1999) Clin. Cardiol. 22(9):595-599), hypercholesterolemia (Kornowski (2003) Coron. Artery Dis. 14(1):61-64), plasma levels of homocysteine (Nagai, et al. (2002) Circ. J. 66(2):158-162), reduced pericardial levels of endostatin (Panchal, et al. (2004) J. Am. Coll. Cardiol. 43(8):1383-1387), certain patterns of inter-individual heterogeneity in hypoxic response (Schultz, et al. (1999) Circulation 100(5):547-552), a haptoglobin phenotype (Hochberg, et al. (2002) Atherosclerosis 161(2):441-446), and C⁵⁸²-T⁵⁸² HIF-1α gene polymorphism (Resar, et al. (2005) Chest 128(2):787-791). Moreover, the levels of adhesion molecules (VCAM-1, ICAM-1, and E-selectin) have been correlated with collateral degree in obstructive coronary artery disease (Guray et al. (2004) Coron. Artery Dis. 15(7):413-7). The biology of collateral growth is under investigation and there is disagreement whether collateral development represents a mere remodeling of the pre-existing (but not hitherto detectable) vasculature or is the result of the de novo arterial growth or vasculogenesis (Helisch, et al. (2003) Microcirculation 10(1):83-97; Schaper and Scholz (2003) Arterioscler. Thromb. Vasc. Biol. 23(7):1143-1151; Simons (2005) Circulation 111(12):1556-156612-14). Temporal patterns of gene expression have been examined in mice after acute hindlimb ischemia (Lee, et al. (2004) J. Am. Coll. Cardiol. 43(3):474-482); however, how the genomic program for collateral vessel development in this animal model relates to humans is unclear.

Therefore, there is a need in the art for biomarkers which can be used in the analysis of collateral development in humans with CAD as well as in the detection of collateral artery development for diagnostic applications. The present invention meets this need in the art.

### SUMMARY OF THE INVENTION

The present invention is a method for determining collateral artery development in a human subject with coronary artery disease. The method involves detecting levels of expression of markers associated with collateral artery development in a test sample from a human subject with coronary artery disease and comparing detected levels with marker levels reference samples, wherein the difference in the levels of expression is indicative of collateral artery development in the human subject. In particular embodiments, the marker is one or more of KLF7, KLF10, KLF11, CREB1, DRAP1, RREB1, RB1, GATA5, RUNX1, RUNX3, CDC42, MYO9B, RAB10, AP3M2, AP3S2, AP4E1, AP4S1, STXBP2, STX6, STX7, TUBA1, H2-ALPHA, TUBA6, TUBB4, TUBB6, BAG4, CARD6, CASP3, CASP10, CUL5, CYCS, IFI16, TNFSF10, SPHK1, EMP1, EMP3, NCK1, PIM1, SCGB3A1, CDKN2D, CDKN2B, ARID4B, MAPKAPK-2, LEPROTL1, INPP4B, GRB2-related 2, ICAM-1, and SSP1.

While some embodiments embrace detecting the levels of nucleic acid molecules encoding the markers, other embodiments embrace detecting the levels of marker proteins. In particular embodiments the marker protein is one or more of sICAM-1, SSP1, Rb1, or Cdc42.

### BRIEF DESCRIPTION OF THE DRAWINGS

FIG. 1 shows a PCA projection of CAD patients resolved over the first and second principal components. Solid circle indicates first cluster containing CAD patients in the score 2 group, whereas dashed circle encompasses the second cluster of CAD patients in the score 0 group. Subjects 1-8 had angiographically confirmed coronary collateral vessels, whereas subjects 9-16 had no angiographically confirmed coronary collateral vessels.

FIG. 2 shows a parallel boxplot analysis of sICAM-1 plasma levels in CAD patients with score 0 and score 2 collateral vessels. Note significantly depressed sICAM-1 plasma levels in CAD patients with score 0 collateral vessels relative to CAD patients with score 2 collateral vessels. Each box contains the middle 50% of its relative data distribution. The horizontal line within each box indicates the median value of each data distribution, whereas the upper and lower horizontal lines of each box represent the 75th and 25th percentiles of each dataset, respectively. The horizontal lines at the ends of the dotted vertical lines indicate maximum and minimum data points.

### DETAILED DESCRIPTION OF THE INVENTION

Studies have suggested that circulating monocytes may play a major role in the collateral formation (Arras, et al. (1998) J. Clin. Invest. 101(1):40-50; Heil, et al. (2002) Am. J. Physiol. Heart Circ. Physiol. 283(6):H2411-2419; van Royen, et al. (2003) Cardiovasc. Res. 57(1):178-185). Abnormalities in monocyte function then may be one of the factors responsible for abnormal collateral growth in certain patient subsets (Schultz, et al. (1999) supra). Therefore, transcriptome profiling of monocytes from patients with CAD was analyzed to determine whether there were detectable differences in the extent of collateral circulating in these patients. To this end, comprehensive characterizations of the molecular determinates of the human CAD monocyte transcriptome was carried out using a combination of established supervised machine learning and knowledge-based algorithms, as well as a gene redundancy reduction microarray bioinformatics data mining technique (Wang, et al. (2005) Bioinformatics 21(8):1530-1537).

The results of this analysis indicated that the monocyte transcriptome of closely matched patients with CAD that possess abundant collateral circulation was significantly different from the transcriptome of collateral-poor CAD subjects. The key differences included significant alterations in transcriptional regulation of specific determinates of monocyte biology that could be directly related to abnormalities in intracellular transport, apoptosis, and cell proliferation. Genes which were differentially expressed between the collateral-rich and collateral-poor CAD subjects are listed in Table 1.

TABLE 1

| | GENBANK | | |
|---|---|---|---|
| | Accession | | GENBANK |
| Gene | No. | Gene | Accession No. |
| KLF7 | NM_003709 | TUBB4 | AK095202 |
| KLF10 | NM_005655 | TUBB6 | NM_032525 |
| KLF11 | AK002186 | BAG4 | BC038505 |
| CREB1 | NM_13442 | CARD6 | NM_032587 |
| DRAP1 | BC018095 | CASP3 | NM_004346 |
| RREB1 | NM_002955 | CASP10 | NM_032977 |
| RB1 | NM_000321 | CUL5 | NM_003478 |
| GATA5 | NM_080473 | CYCS | NM_018947 |
| RUNX1 | X90980 | IFI16 | AK094968 |
| RUNX3 | NM_004350 | TNFSF10 | AI376429 |
| CDC42 | NM_044472 | SPHK1 | NM_021972 |
| MYO9B | L29149 | EMP1 | NM_001423 |
| RAB9A | NM_004251 | EMP3 | NM_001425 |
| RAB10 | AF297660 | NCK1 | NM_006153 |
| AP3M2 | NM_006803 | PIM1 | NM_002648 |
| AP3S2 | NM_005829 | SCGB3A1 | NM_052863 |
| AP4E1 | BC015224 | CDKN2D | NM_001800 |
| AP4S1 | AB030654 | CDKN2B | NM_078487 |
| STXBP2 | NM_006949 | ARID4B | NM_016374 |
| STX6 | NM_005819 | MAPKAPK-2 | NM_004759 |
| STX7 | NM_003569 | LEPROTL1 | NM_015344 |
| TUBA1 | NM_006000 | INPP4B | NM_003866 |
| H2-ALPHA | AK093116 | GRB2-related 2 | NM_004810 |
| TUBA6 | NM_032704 | ICAM-1 | J03132 |
| | | SSP1 | NM_001040058 |

The collateral-rich and collateral-poor patient populations in the study were carefully matched for all known CAD and collateral development risk factors, with the only significant difference being the angiographic extent of CAD as documented by Gensini and vessel scoring systems. The severity of CAD has been considered a predictor of collateral development (Koerselman, et al. (2003) supra; Schaper and Scholz (2003) supra; Fulton (1965) The coronary arteries; arteriography, microanatomy, and pathogenesis of obliterative coronary artery disease. Springfield, Ill.: C. C. Thomas). Another important determinant of the collateral presence is thought to be the gradual development of coronary stenosis rather then a sudden coronary occlusion (Fujita, et al. (1999) supra; Schaper and Scholz (2003) supra). To evaluate the effect of differences in CAD severity, additional analysis was carried out to determine the contribution of the differences in CAD extent to the gene ontology (GO) class memberships describing differences in monocyte transcriptome. Re-ordering the data set into two classes based on the angiographic extent of CAD resulted in the loss of significant GO feature terms associated with collateral class, indicating the presence of two distinct monocyte transcriptional processes which occur in these patients. This also indicates that angiographic CAD extent does not substantially influence transcriptional regulation of coronary collateralization.

The results presented herein provide “collateralgenic” monocyte transcription profiles in patients with CAD, which are independent of the angiographic extent of CAD. Thus, the present invention relates to the molecular analysis of coronary collateralization and provides methods for obtaining information about consistent molecular alterations that advance both the understanding of the basic biology of coronary collateral artery formation as well as the clinically relevant aspects of coronary collateralization in coronary artery disease. In particular, the present invention provides a plurality of nucleic acid molecules and proteins and molecular profiles which serve as markers for determining collateral artery formation in coronary artery disease.

Coronary collateralization markers according to the present invention include any nucleic acid sequence or molecule or corresponding polypeptide encoded by the nucleic acid sequence or molecule which demonstrates altered expression (i.e., higher or lower expression) in collateral-rich (e.g., collateral score of 1, 2 or 3) coronary artery disease samples relative to collateral-poor (e.g., collateral score of 0) coronary artery disease samples. Coronary collateralization markers of the present invention include, KLF7 (ubiquitin Kruppel-like transcription factor), KLF10 (Kruppel-like factor 10/TGFB inducible early growth response), KLF11 (Kruppel-like factor 11/TGFB inducible early growth response 2), CREB1 (cAMP-responsive element binding protein 1), DRAP1 (DR1-associated protein 1 (negative cofactor 2 alpha)), RREB1 (ras-responsive element binding protein 1), RB1 (Retinoblastoma 1), GATA5 (GATA binding protein 5), RUNX1 (Runt-related transcription factor 1), RUNX3 (Runt-related transcription factor 3), CDC42 (Cell division cycle 42 (GTP binding protein, 25 kDa)), MYO9B (Myosin IXB), RAB9A (RAB9A, member RAS oncogene family), RAB10 (RAB10, member RAS oncogene family), AP3M2 (Adaptor-related protein complex 3, mu 2 subunit), AP3S2 (Adaptor-related protein complex 3, sigma 2 subunit), AP4E1 (Adaptor-related protein complex 4, epsilon 1 subunit), AP4S1 (Adaptor-related protein complex 4, sigma 1 subunit), STXBP2 (Syntaxin binding protein 2), STX6 (Syntaxin 6), STX7 (Syntaxin 7), TUBA1 (Tubulin, alpha (testis specific)), H2-ALPHA (Alpha-tubulin isotype H2-alpha), TUBA6 (Tubulin, alpha 6), TUBB4 (Tubulin, beta 4), TUBB6 (Tubulin, beta 6), BAG4 (CL2-associated athanogene 4), CARD6 (Caspase recruitment domain family, member 6), CASP3 (Caspase 3, apoptosis-related cysteine peptidase), CASP10 (Caspase 10, apoptosis-related cysteine peptidase), CUL5 (Cullin 5), CYCS (Cytochrome c, somatic), IFI16 (Interferon, gamma-inducible protein 16), TNFSF10 (Tumor necrosis factor (ligand) superfamily, member 10), SPHK1 (Sphingosine kinase 1), EMP1 (Epithelial membrane protein 1), EMP3 (Epithelial membrane protein 3), NCK1 (NCK adaptor protein 1), PIM1 (Pim-1 oncogene), SCGB3A1 (Secretoglobin, family 3A, member 1), CDKN2D (Cyclin-dependent kinase inhibitor 2D; p19, inhibits CDK4), CDKN2B (Cyclin-dependent kinase inhibitor 2B; p15, inhibits CDK4), ARID4B (retinoblastoma binding protein 1-like 1), MAPKAPK-2 (mitogen-activated protein kinase-activated protein kinase 2), LEPROTL1 (leptin receptor overlapping transcript-like 1), INPP4B (inositol polyphosphate-4-phosphatase, type II), GRB2-related 2 (growth factor receptor-binding protein), ICAM-1 (intracellular adhesion molecule-1, and SSP1 (osteopontin). See Table 1. In particular embodiments, at least one marker is employed in the instant method. In other embodiments, at least two, three, four, five, six, seven, eight, nine, ten, or more markers are employed in the instant method. In one embodiment one or more markers employed in the instant method include ARID4B, MAPKAPK-2, LEPROTL1, INPP4B, GRB2-related 2, CDC42, RB1, ICAM-1, or SSP1. In a particular embodiment, one or more markers employed in the instant method include CDC42, RB1, ICAM-1, or SSP1.

Nucleic acids according to the present invention can include any polymer or oligomer of pyrimidine and purine bases, preferably cytosine, thymine, and uracil; and adenine and guanine, respectively. See Lehninger (1982) Principles of Biochemistry, at pages 793-800. The present invention contemplates any deoxyribonucleotide, ribonucleotide or peptide nucleic acid component, and any chemical variants thereof, such as methylated, hydroxymethylated or glucosylated forms of these bases, and the like. The polymers or oligomers can be heterogeneous or homogeneous in composition. In addition, the nucleic acids may be DNA or RNA, or a mixture thereof, and can exist permanently or transitionally in single-stranded or double-stranded form, including homoduplex, heteroduplex, and hybrid states. Oligonucleotide and polynucleotide are included in this definition and relate to two or more nucleic acids in a polynucleotide.

Gene expression monitoring is well-known in the art as being useful for distinguishing between cells that express different phenotypes. In accordance with the present invention, gene expression monitoring is used to determine collateral artery development in coronary artery disease patients thereby providing a means to identify patients with a more favorable prognosis or an enhanced likelihood of response to therapeutic angiogenesis agents.

In a particular embodiment, collateral artery development in CAD subjects is determined by gene expression profile analysis. As used herein, an “expression profile” is a measurement of the relative abundance of a plurality of cellular constituents. Such measurements can include RNA or protein abundances or activity levels. An expression profile involves providing a pool of target nucleic acid molecules or polypeptides, hybridizing the pool to an array of probes immobilized on predetermined regions of a surface, and quantifying the hybridized nucleic acid molecules or proteins. The expression profile can be a measurement, for example, of the transcriptional state or the translational state of the cell. See U.S. Pat. Nos. 6,040,138; 6,013,449; and 5,800,992, which are hereby incorporated by reference in their entireties.

An array is used herein to describe a solid support with peptide or nucleic acid probes attached to said support. Arrays typically contain a plurality of different nucleic acid or peptide probes that are coupled to a surface of a substrate in different, known locations. These arrays, also described as “microarrays” or colloquially “chips” have been generally described in the art, for example, U.S. Pat. Nos. 5,143,854; 5,445,934; 5,744,305; 5,677,195; 6,040,193; 5,424,186 and Fodor, et al. (1991) Science 251:767-777. These arrays can generally be produced using mechanical synthesis methods or light-directed synthesis methods which incorporate a combination of photolithographic methods and solid-phase synthesis methods. Techniques for the synthesis of these arrays using mechanical synthesis methods are described in, e.g., U.S. Pat. Nos. 5,384,261 and 6,040,193. Although a planar array surface is preferred, the array can be fabricated on a surface of virtually any shape or even a multiplicity of surfaces. Arrays can be peptides or nucleic acids on beads, gels, polymeric surfaces, fibers such as fiber optics, glass or any other appropriate substrate, see U.S. Pat. Nos. 5,770,358; 5,789,162; 5,708,153; 6,040,193 and 5,800,992.

The transcriptional state of a sample refers to the identities and relative abundances of the RNA species, especially mRNAs present in the sample. Preferably, a substantial fraction of all constituent RNA species in the sample are measured, but at least a sufficient fraction is measured to characterize the state of the sample. The transcriptional state can be conveniently determined by measuring transcript abundances by any of several existing gene expression technologies as disclosed herein. On the other hand, translational state refers to the identities and relative abundances of the constituent protein species in the sample. As is known to those of skill in the art, the transcriptional state and translational state are related.

For the purposes of the present invention, a gene expression monitoring system can include a nucleic acid probe array (such as those described above), membrane blot (such as used in hybridization analysis such as northern, Southern, or dot blot analysis, and the like), microwells, sample tubes, gels, beads or fibers (or any solid support containing bound nucleic acids). See U.S. Pat. Nos. 5,770,722; 5,874,219; 5,744,305; 5,677,195; 5,445,934; and 5,800,992.

A gene expression monitoring system according to the present invention can be used to facilitate a comparative analysis of expression in different cells or tissues, different subpopulations of the same cells or tissues, different physiological states of the same cells or tissue, or different cell populations of the same tissue.

The term differentially expressed as used herein means that a measurement of a cellular constituent varies in two samples. The cellular constituent can be either upregulated in the test sample relative to the reference sample or downregulated in the test sample relative to the reference sample. See U.S. Pat. No. 5,800,992.

One of skill in the art will appreciate that it is desirable to have samples containing target nucleic acid sequences that reflect the transcripts of interest. Therefore, suitable samples can contain transcripts of interest or can alternatively contain nucleic acids derived from the transcripts of interest. As used herein, a nucleic acid derived from a transcript refers to a nucleic acid for whose synthesis the mRNA transcript or a subsequence thereof has ultimately served as a template. Thus, a cDNA reverse transcribed from a transcript, an RNA transcribed from that cDNA, a DNA amplified from the cDNA, an RNA transcribed from the amplified DNA, etc., are all derived from the transcript and detection of such derived products is indicative of the presence and/or abundance of the original transcript in a sample. Thus, suitable samples include, but are not limited to, transcripts of the gene or genes, cDNA reverse transcribed from the transcript, cRNA transcribed from the cDNA, DNA amplified from the genes, RNA transcribed from amplified DNA, and the like.

Transcripts, as used herein, can include, but are not limited to pre-mRNA nascent transcript(s), transcript processing intermediates, mature mRNA(s) and degradation products. It is not necessary to monitor all types of transcripts to practice this invention. For example, one may choose to practice the invention to measure the mature mRNA levels only.

In one embodiment, a sample is a homogenate of cells (e.g., blood cells), tissues or other biological samples obtained from a subject with coronary artery disease. In particular embodiments, the sample contains monocytes. Preferably, such sample is a nucleic acid preparation, e.g., a total RNA preparation of a biological sample. More particularly, some embodiments embrace a sample containing the total mRNA isolated from a biological sample. Those of skill in the art will appreciate that the total mRNA prepared with most methods includes not only the mature mRNA, but also the RNA processing intermediates and nascent pre-mRNA transcripts. For example, total mRNA purified with a poly (T) column contains RNA molecules with poly (A) tails. Those poly A+ RNA molecules could be mature mRNA, RNA processing intermediates, nascent transcripts or degradation intermediates.

Biological samples can be of any biological tissue or fluid or cells. Frequently the sample will be a “clinical sample” which is a sample derived from a patient. Clinical samples provide rich sources of information regarding the various states of genetic network or gene expression. Typical clinical samples include, but are not limited to, sputum, blood, blood cells, tissue or fine needle biopsy samples, urine, peritoneal fluid, and pleural fluid, or cells therefrom. Biological samples can also include sections of tissues such as frozen sections taken for histological purposes.

A subject with coronary artery disease can be identified based upon one or more well-known clinical criteria including, e.g., increased LDL levels, hypertension, hyperlipidemia, increased triglyceride levels, angina, and a family history of coronary artery disease. In general, subjects with coronary artery disease have atheromatous plaques that cause obstruction of blood vessels. As the plaques grow in thickness and obstruct more than 70 percent of the diameter of the vessel, the subject develops symptoms of obstructive coronary artery disease. At this stage of the disease process, the patient can be said to have ischemic heart disease. The symptoms of ischemic heart disease are often first noted during times of increased workload of the heart. For instance, the first symptoms include exertional angina or decreased exercise tolerance. As the degree of coronary artery disease progresses, there may be near-complete obstruction of the lumen of the coronary artery, severely restricting the flow of oxygen-carrying blood to the myocardium. Individuals with this degree of coronary heart disease typically have suffered from one or more myocardial infarctions (MI), and may have signs and symptoms of chronic coronary ischemia, including symptoms of angina at rest and flash pulmonary edema.

In one embodiment, the level of expression of a marker for collateral artery development in a subject with coronary artery disease is assessed by detecting the presence of a nucleic acid corresponding to the marker in the sample. In another embodiment, the level of expression of a marker for collateral artery development is assessed by detecting the presence of a protein corresponding to the marker in the sample. In one aspect, the presence of the protein is detected using a reagent which specifically binds to the protein, e.g., an antibody, an antibody derivative, and/or an antibody fragment.

Detection involves contacting a biological sample with a compound or an agent capable of detecting a marker associated with collateral artery development such that the presence of the marker is detected in the biological sample. An example of an agent for detecting marker RNA is a labeled nucleic acid probe capable of hybridizing to marker RNA. The nucleic acid probe can be, for example, complementary to any of the nucleic acid markers of collateral artery development disclosed herein, or a portion thereof, such as an oligonucleotide which specifically hybridizes marker RNA. The term probe, as defined herein, is meant to encompass oligonucleotides from ten to twenty-five base pairs in length, but longer sequences can be employed. Probes, while perhaps capable of priming, are designed for hybridizing to the target DNA or RNA and need not be used in an amplification process.

An example of an agent for detecting a marker protein is a labeled antibody capable of binding to the marker protein. Antibodies can be polyclonal, or more desirably, monoclonal. An intact antibody, antibody derivative, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

Suitable primers, probes, or oligonucleotides useful for gene expression analysis are exemplified herein or can be generated by the skilled artisan from marker sequences provided by GENBANK or EMBL databases or the like. See Table 1.

The detection methods described herein can be used to detect marker RNA or marker protein in a biological sample in vitro as well as in vivo. In vitro techniques for detection of marker RNA include, but are not limited to, northern hybridization and in situ hybridization. In vitro techniques for detection of marker protein include, but are not limited to, enzyme-linked immunosorbent assays (ELISAs), western blots, immunoprecipitations, and immunofluorescence assays. Alternatively, a marker protein can be detected in vivo in a subject by introducing into the subject a labeled antibody against the marker protein. For example, the antibody can be labeled with a radioactive marker whose presence and location in a subject can be detected by standard imaging techniques.

One of skill in the art would appreciate that it is desirable to inhibit or destroy RNase present in homogenates before homogenates can be used for hybridization. Methods of inhibiting or destroying nucleases are well-known in the art. In some embodiments, cells or tissues are homogenized in the presence of chaotropic agents to inhibit nuclease. In some other embodiments, RNases are inhibited or destroyed by heat treatment followed by proteinase treatment.

Methods of isolating total mRNA are also well-known to those of skill in the art. For example, methods of isolation and purification of nucleic acids are described in detail in Chapter 3 of Laboratory Techniques in Biochemistry and Molecular Biology: Hybridization with Nucleic Acid Probes, Part I. Theory and Nucleic Acid Preparation, P. Tijssen, ed. Elsevier, N.Y. (1993).

In certain embodiments, total RNA is isolated from a given sample using, for example, an acid guanidinium-phenol-chloroform extraction method followed by polyA+ mRNA isolation by oligo dT column chromatography or by using (dT)n magnetic beads (see, e.g., Sambrook et al. (1989) Molecular Cloning: A Laboratory Manual (2nd ed.), Vols. 1-3, Cold Spring Harbor Laboratory; Current Protocols in Molecular Biology (1987) Ausubel et al., ed. Greene Publishing and Wiley-Interscience, New York). See also PCT/US99/25200 for complexity management and other sample preparation techniques.

Frequently, it is desirable to amplify the nucleic acid sample prior to hybridization. One of skill in the art will appreciate that methods of amplifying nucleic acids are well-known in the art and that whatever amplification method is used, if a quantitative result is desired, care must be taken to use a method that maintains or controls for the relative frequencies of the amplified nucleic acids to achieve quantitative amplification. Methods of quantitative amplification are well-known to those of skill in the art. For example, quantitative PCR involves simultaneously co-amplifying a known quantity of a control sequence using the same primers. This provides an internal standard that can be used to calibrate the PCR reaction. A high-density array can then be performed which includes probes specific to the internal standard for quantification of the amplified nucleic acid. Other suitable amplification methods include, but are not limited to polymerase chain reaction (PCR) (Innis, et al. (1990) PCR Protocols. A guide to Methods and Application. Academic Press, Inc., San Diego), ligase chain reaction (LCR) (see Wu and Wallace (1989) Genomics 4:560; Landegren, et al. (1988) Science 241:1077; Barringer, et al. (1990) Gene 89:117), transcription amplification (Kwoh, et al. (1989) Proc. Natl. Acad. Sci. USA 86:1173), and self-sustained sequence replication (Guatelli, et al. (1990) Proc. Nat. Acad. Sci. USA 87:1874).

Cell lysates or tissue homogenates often contain a number of inhibitors of polymerase activity. Therefore, the skilled practitioner typically incorporates preliminary steps to isolate total RNA or mRNA for subsequent use as an amplification template. One tube mRNA capture methods can be used to prepare poly(A)+ RNA samples suitable for immediate RT-PCR in the same tube (Boehringer Mannheim). The captured mRNA can be directly subjected to RT-PCR by adding a reverse transcription mix and, subsequently, a PCR mix.

In one embodiment, the sample mRNA is reverse transcribed with a reverse transcriptase and a primer consisting of oligo dT and a sequence encoding the phage T7 promoter to provide single stranded DNA template. The second DNA strand is polymerized using a DNA polymerase. After synthesis of double-stranded cDNA, T7 RNA polymerase is added and RNA is transcribed from the cDNA template. Successive rounds of transcription from each single cDNA template results in amplified RNA. Methods of in vitro polymerization are well-known to those of skill in the art (see, e.g., Sambrook, supra).

As one of skill in the art can appreciate, the direct transcription method described above provides an antisense RNA (aRNA) pool. Where aRNA is used as the target nucleic acid, the oligonucleotide probes provided in the array are chosen to be complementary to subsequences of the antisense nucleic acids. Conversely, where the target nucleic acid pool is a pool of sense nucleic acids, the oligonucleotide probes are selected to be complementary to subsequences of the sense nucleic acids. Finally, where the nucleic acid pool is double-stranded, the probes can be of either sense as the target nucleic acids include both sense and antisense strands.

The generation of either sense or antisense nucleic acid molecules can be achieved using a variety of methods. For example, the cDNA can be directionally cloned into a vector (e.g., pBLUSCRIPT II KS (+) phagemid) such that it is flanked by the T3 and T7 promoters. In vitro transcription with the T3 polymerase will produce RNA of one sense (the sense depending on the orientation of the insert), while in vitro transcription with the T7 polymerase will produce RNA having the opposite sense. Other suitable cloning systems include phage lambda vectors designed for Cre-loxP plasmid subcloning (see, e.g., Palazzolo, et al. (1990) Gene 88:25-36).

Gene expression analysis can be achieved using a variety of alternative methods or combinations of methods including, e.g., quantitative PCR, electrochemical denaturation of double-stranded nucleic acid molecules (U.S. Pat. Nos. 6,045,996 and 6,033,850), the use of multiple arrays (arrays of arrays; U.S. Pat. No. 5,874,219), the use of scanners to read the arrays (U.S. Pat. Nos. 5,631,734; 5,744,305; 5,981,956 and 6,025,601), methods for mixing fluids (U.S. Pat. No. 6,050,719), integrated device for reactions (U.S. Pat. No. 6,043,080), integrated nucleic acid diagnostic device (U.S. Pat. No. 5,922,591), and nucleic acid affinity columns (U.S. Pat. No. 6,013,440).

The invention also encompasses kits for assessing collateral artery development in coronary artery disease. The kit can contain a labeled compound or agent capable of detecting collateral artery markers (e.g., nucleic acid markers and/or protein markers) in a biological test sample, a means for determining the amount of collateral artery markers in the test sample, and a means for comparing the amount of collateral artery markers in the test sample with a reference sample. The compound or agent can be packaged in a suitable container. The kit can further contain instructions for using the kit to detect collateral artery markers.

As used herein, a reference sample can be a sample with a known collateral score (e.g., 0, 1, 2, 3) for which there is a known level of expression of a collateral biomarker (e.g., one or more marker listed in Table 1). Those skilled in the art will recognize that expression profiles from one or more reference samples can be input to a database. A relational database is preferred and can be used, but one of skill in the art will recognize that other databases could be used. A relational database is a set of tables containing data fitted into predefined categories. Each table, or relation, contains one or more data categories in columns. Each row contains a unique instance of data for the categories defined by the columns. For example, a typical database for the invention would include a table that describes a sample with columns for age, gender, reproductive status, expression profile and so forth. Another table would describe a disease: symptoms, level, sample identification, expression profile and so forth. See U.S. Pat. No. 6,185,561.

In one embodiment the invention matches the test sample to a database of reference samples. The database is assembled with a plurality of different samples to be used as reference samples. An individual reference sample in one embodiment will be obtained from a patient during a visit to a medical professional. The sample could be, for example, a tissue, blood, urine, or saliva sample. Information about the physiological, disease and/or pharmacological status of the sample will also be obtained through any method available. This may include, but is not limited to, expression profile analysis, clinical analysis, medical history and/or patient interview. For example, the patient could be interviewed to determine age, sex, ethnic origin, symptoms or past diagnosis of disease, and the identity of any therapies the patient is currently undergoing. A plurality of these reference samples will be taken. A single individual can contribute a single reference sample or more than one sample over time. One skilled in the art will recognize that confidence levels in predictions based on comparison to a database increase as the number of reference samples in the database increases. One skilled in the art will also recognize that some of the indicators of status will be determined by less precise means, for example information obtained from a patient interview is limited by the subjective interpretation of the patient.

The database is organized into groups of reference samples. Each reference sample contains information about physiological, pharmacological and/or disease status. For example, the database can be a relational database with data organized in three data tables, one where the samples are grouped primarily by physiological status, one where the samples are grouped primarily by disease status, and one where the samples are grouped primarily by pharmacological status. Within each table the samples can be further grouped according to the two remaining categories. For example, the physiological status table could be further categorized according to disease and pharmacological status.

As will be appreciated by one of skill in the art, the present invention can further include data analysis systems, methods, analysis software and etc. For example, a computer system for analyzing physiological states, levels of disease states and or therapeutic efficacy can be employed. In general, the computer system can include a processor, and memory coupled to said processor which encodes one or more programs. The programs encoded in memory cause the processor to perform method steps, wherein the expression profiles and information about physiological, pharmacological and disease states are received by the computer system as input. U.S. Pat. No. 5,733,729 illustrates an example of a computer system that can be used to execute data analysis software. Computer systems suitable for use with the invention can also be embedded in a measurement instrument. The embedded systems can control the operation of, for example, a GENECHIP Probe array scanner (also called a GENEARRAY scanner sold by AGILENT corporation, Palo Alto, Calif.) as well as executing computer codes.

Computer methods can be used to measure the variables and to match samples to eliminate gene expression differences that are a result of differences that are not of interest. For example, a plurality of values can be input into computer code for one or more physiological, pharmacological and/or disease states. The computer code can thereafter measure the differences or similarities between the values to eliminate changes not attributable to a value of interest. Examples of computer programs and databases that can be used for this purpose are shown in U.S. Pat. Nos. 6,185,561 and 6,600,996). Computer software to analyze data generated by microarrays is commercially available from AFFYMETRIX Inc. (Santa Clara, Calif.) as well as other companies. Other databases can be constructed using the standard database tools available from MICROSOFT (e.g., EXCEL and ACCESS).

The invention is an improvement in the art in that it provides a reliable method for detecting collateral artery development in CAD subjects. The instant method finds application in CAD prognosis as well as in providing predictive information pertaining to the likelihood of response to therapeutic angiogenesis agents.

The invention is described in greater detail by the following non-limiting examples.

### Example 1

### Patient Selection

Patients over the age of 18, undergoing diagnostic coronary angiography were eligible for entry into the study. Patients were excluded if they had other conditions thought to influence potential neovascularization such as symptomatic peripheral arterial disease, recent ST segment elevation myocardial infarction (<72 hours before enrollment), increased white blood counts, or a known malignancy within 5 years. Cardiac history and risk factors were documented together with any data known to influence collateral growth including the use of medication (Klauber, et al. (1996) Circulation. 94(10):2566-2571; Panet, et al. (1994) J. Cell Physiol. 158(1):121-127; Volpert, et al. (1996) J. Clin. Invest. 98(3):671-679), age (Rivard, et al. (1999) Circulation 99(1):111-120), hypercholesterolemia (Van Belle, et al. (1997) Circulation 96(8):2667-2674), diabetes (Waltenberger (2001) Cardiovasc. Res. 49(3):554-560) and smoking (Melkonian, et al. (2002) Toxicol. Sci. 68(1):237-248). Only patients who had angiographically evident coronary artery disease, and absent (score 0) or well-developed (score 2) collateral circulations were included in this analysis.

The groups were not statistically different in regards to age, CAD risk factors (including weight and diabetic status), clinical presentation, indications for coronary angiography, total cholesterol and LDL levels or past coronary revascularization procedures (Table 2).

TABLE 2

| | Collateral | Collateral | |
|---|---|---|---|
| | Score 2 | Score 0 | P |
| Variable | (n = 8) | (n = 0) | Value |
| Age, year | 61 ± 9 | 57 ± 7.8 | 0.38 |
| Men, n | 8 | 8 | 1.0 |
| Height, meters | 1.75 ± 0.05 | 1.76 ± 0.05 | 0.69 |
| Weight, kilogram | 91 ± 20 | 99 ± 26 | 0.49 |
| BSA, m² | 2.06 ± 0.22 | 2.14 ± 0.25 | 0.49 |
| BMI | 30 ± 6 | 32 ± 8 | 0.53 |
| Hypertension, n | 7 | 6 | 0.52 |
| Hyperlipidemia, n | 8 | 6 | 0.13 |
| Total Cholesterol, mg/dL | 173 ± 36 | 173 ± 4 | 0.98 |
| LDL, mg/dL | 96 ± 43 | 101 ± 12 | 0.82 |
| HDL, mg/dL | 47 ± 14 | 47 ± 6 | 0.77 |
| Triglycerides, mg/dL | 198 ± 106 | 148 ± 41 | 0.40 |
| Diabetes, n | 3 | 1 | 0.25 |
| Insulin-Dependent, n | 1 | 0 | 0.30 |
| Smoking status, n | | | 0.82 |
| Current | 3 | 3 | |
| Ex-smoker | 3 | 2 | |
| Never smoked | 2 | 3 | |
| Family history of CAD, n | 4 | 3 | 0.61 |
| Previous MI, n | 2 | 2 | 1.0 |
| Previous PCI, n | 2 | 2 | 1.0 |
| Previous CABG, n | 2 | 1 | 0.52 |
| Vessel Score | 2.4 ± 0.92 | 1.1 ± 0.12 | 0.003 |
| Gensini Score | 50 ± 22 | 23 ± 20 | 0.02 |
| >50% LM stenosis, n | 0 | 1 | 0.30 |
| Ejection fraction <35% | 0 | 0 | 1.0 |
| Indication of angiography, n | | | 0.29 |
| Stable angina | 6 | 6 | |
| Unstable angina, troponin, - | 1 | 2 | |
| NSTEMI | 1 | 0 | |
| Medications, n | | | |
| Aspirin | 8 | 6 | 0.13 |
| Clopidigrel | 1 | 1 | 1.0 |
| ACE-I | 4 | 4 | 1.0 |
| ARB | 1 | 1 | 1.0 |
| Beta-blockers | 8 | 6 | 0.13 |
| Calcium channel blockers | 3 | 1 | 0.25 |
| Diuretics | 2 | 2 | 1.0 |
| Digitalis | 0 | 1 | 0.30 |
| Lipid lowering therapy | 7 | 7 | 1.0 |
| Nitrates | 2 | 0 | 0.13 |
| Analgesics | 2 | 1 | 0.52 |
| Glycoprotein IIb/IIIa inhib. | 1 | 0 | 0.31 |
| Anticoagulants | 1 | 1 | 1.0 |

### Example 2

### Coronary Angiography and Collateral Scoring

Severity of coronary artery disease on X-ray angiography was estimated using a vessel score, defined as the number of vessels with at least one 50% stenosis, and the Gensini scoring system (Gensini (1983) Am. J. Cardiol. 51(3):606). Coronary collateral extent was assessed based on a modified Rentrop scoring system (Schultz, et al. (1999) supra). Angiograms were reviewed by an experienced angiographer and then by a separate angiographer blinded to the initial reading. In cases of disagreement, the angiograms were reviewed by a third angiographer blinded to the initial two readings. Clinical and angiographic data were not revealed to those involved in gene expression or monocyte analysis. Left ventricular function was estimated by left ventriculography at the time of cardiac catheterization or by echocardiography performed during the same hospitalization. A total of 100 ml of blood was collected from the side arm of the introducer sheath in the femoral artery prior to angiography and immediately processed for monocytes isolation as described herein.

### Example 3

### Human Monocyte Cell Separation

Human monocytes were separated from whole blood by standard procedures (Ouyang, et al. (2000) Immunity 12(1):27-37). Briefly, peripheral blood mononuclear cells were isolated by FICOLL density gradient centrifugation and then used immediately for monocyte isolation by positive selection with CD14 antibody-coated microbeads. Cells were then separated using AUTOMACS with the positive selection protocol and cell collections were made from both positive and negative ports. Stained aliquots of the positive and negative cell fractions were collected and analyzed by flow cytometry to assess purity.

### Example 4

### Human Monocyte RNA Extraction, Target Processing and Labeling

Labeled cRNAs were generated using the low RNA input fluorescent linear amplification kit (AGILENT). All samples were labeled with cyanine 5 and a reference cRNA was generated and labeled with cyanine 3. To generate a reference cRNA, 500 ng of total RNAs from each control sample were mixed and 500 ng mixed total RNA was amplified and labeled with Cy3 (4 reactions were carried out to generate enough Cy3 NC for all 16 hybridizations). The hybridizations for each sample were performed using an AGILENT in situ hybridization kit. For each hybridization, 0.75 μg Cyanine 5-labeled, linearly amplified cRNA from each sample was mixed with an equal amount of Cyanine 3-labeled, linearly amplified reference cRNA. The mixed cRNA was fragmented by incubation with the fragmentation buffer at 60° C. for 30 minutes. The equal amount of 2× hybridization buffer was added to the fragmented cRNA mixture and hybridized to AGILENT human whole genome oligo array (G4112A) at 60° C. for 17 hours. Fluorescent images of hybridized microarrays were obtained using an AGILENT DNA Microarray scanner, analyzed with AGILENT Feature Extraction software and the data was stored in a database.

### Example 5

### Statistical Analyses

Clinical results are reported as mean±standard deviation. Analysis between groups for statistically significant differences in categorical data was performed using the χ² test and for continuous variables using the t-test (STATA; StataCorp, College Station, Tex.).

The primary human monocyte dataset was composed of gene expression data from 16 patients, 8 patients with well-developed collateral vessels (score 2) and 8 patients with no collateral vessels (score 0). To assess the possible confounding effects of disease severity, these same subjects were regrouped according to percent or degree occlusion of one or more of the coronary vessels to form a secondary dataset. In this secondary dataset patients with 2 to 3 vessel disease (n=7) were compared to patients with 1 vessel disease (n=9). The two datasets, the first based on collateral vessel scores and the second based on disease severity, were further filtered based on critical p values (p≦0.05 and 0.01) as assessed between subjects Welch approximation of unequal group variances. Significance analysis of microarrays (Tusher, et al. (2001) Proc. Natl. Acad. Sci. USA 98(9):5116-5121) was used determine an approximate false discovery rate of 32% for the primary patient feature selection set.

To improve the accuracy of the 4 statistical classifiers used herein, small subsets of top-ranked class membership predictors were generated using the HykGene classification method (Wang, et al. (2005) Bioinformatics 21(8):1530-1537). GO::TermFinder software available from Princeton University was used to classify data according to biological process, molecular function, and cellular component. Chilibot text mining software was used to assess known relationships between angiogenesis and statistically significant differentially expressed genes within the enriched biological process terms of the GO analysis (Chen and Sharp (2004) BMC Bioinformatics 5:147).

### Example 6

### Quantitative PCR Validation

Total monocyte RNA was isolated and cDNA synthesized as described herein. PCR amplification was carried out with gene-specific TAQMAN-based assays (APPLIED BIOSYSTEMS, Foster City, Calif.) on a GENEAMP 5700 sequence detection system (APPLIED BIOSYSTEMS): LEPROTL1 (NM_{—}015344; Hs00209745_m1), MAPKAPK-2 (NM_{—}004759; Hs00358962_m1), GRB2-related Protein (NM_{—}004810; Hs00191325_m1), Inositol Polyphosphate-4-Phosphatase (NM_{—}003866;Hs00182580_m1), ARID4B (NM_{—}016374; Hs00249610_m1), and normalized to ACTB (NM_{—}001615.3;Hs00242273_m1). Relative gene expression was assessed by the 1^{-ΔΔCT} method. Statistical significance was assessed in R with one sample Wilcoxon signed rank or one sample t-tests.

### Example 7

### Protein Expression Analysis

ELISA Analysis. Human sICAM-1 was analyzed in heparinized plasma using a sICAM-1 ELISA kit (R&D Systems, Inc., Minneapolis, Minn.) in 100 μl of diluted plasma (1:20) that was incubated on the ELISA plate for 1.5 hours, followed by washing and incubation with secondary reagents. Plates were read using a Multiskan Microplate Spectrophotometer (Thermo Electron, Waltham, Mass.).

Western Blot Analysis. Peripheral blood mononuclear cells from collateral score 0 and 2 patients were pelleted at 4° C. and resuspended in 200 μl of RIPA buffer. Ten micrograms of protein from each patient was resolved on a SDS-PAGE gel, transferred to a PVDF membrane and probed with a polyclonal anti-Cdc42 antibody (Cell Signaling, Danvers, Mass.) overnight at 40° C. The membrane was incubated with a anti-rabbit antibody conjugated to horseradish peroxidase and detected using the PHOTOTOPE HRP Western Blot Detection System (Cell Signaling). Human serum albumin (HSA) was used as a lane loading marker. Membranes containing plasma proteins were incubated overnight at 40° C. with a monoclonal antibody to HSA (Sigma Chemical Corp., St. Louis, Mo.). The secondary antibody and detection conditions were the same as described for Cdc42.

### Example 8

### Gene Expression Profile Analysis

To demonstrate the role of monocytes in collateral development in patients with CAD, sixteen patients with angiographically evident disease, grouped based on their degree of angiographically detectable collateral circulation, were evaluated. The score 2 group was composed of 8 patients with well-developed coronary collaterals and the score 0 group, of 8 patients with no angiographically evident collateral circulations. The groups were not statistically different (Table 2), with the only observed difference between groups being the extent of CAD measured by a vessel score and Gensini score (score 2 vs. score 0 collaterals groups: 2.4±0.9 vs. 1.1±0.12 diseased vessels (p=0.003), and 50±23 vs. 23±20, Gensini score (p=0.02)).

AGILENT (Santa Clara, Calif.) human whole genome oligonucleotide arrays (G4112A) containing 44,000 features, representing 33,000 unique genes, were profiled using total RNA extracted from peripheral blood monocytes. Two subsets of transcripts demonstrating statistically robust differences (p≦0.05 and 0.01) in abundance between patient groups were identified. An inclusive subset of 1327 transcripts (p≦0.05) (S1) was used for GO analysis, while a more statistically restricted (p≦0.01) subset composed of 256 transcripts (S2), was used as a feature set with the aim of predicting patient class membership via the redundancy-based HykGene classification method (Wang, et al. (2005) supra). The hybrid HykGene classification system directly addresses the large number of features and the relatively small number of samples which give rise to statistical concerns in classification analysis of gene expression data due.

The GO analysis (Table 3) showed that after correction for multiple hypothesis testing, there were statistically significant enrichments of transcripts within the S1 subset displaying transcriptional activator and transcription cofactor activities. These differences in molecular function were also observed as significant enrichments of transcripts involved in the biological processes of transcription, cell organization and biogenesis, cellular localization, and intracellular transport, response to stress, apoptosis, and cell proliferation.

TABLE 3

| | | | | |
|---|---|---|---|---|
| | | | Corrected | Number of |
| GO Term | GOID | P Value | P Value | Annotations |

Biological Process

| | | | | |
|---|---|---|---|---|
| Transcription | GO:0006350 | 1.5e−10 | 1.5e−10 | 125/3195 |
| Cell Organization and | GO:0016043 | 8.6e−09 | 8.6e−06 | 84/1985 |
| Biogenesis | | | | |
| Cellular Localization | GO:0051641 | 4.0e−06 | 0.004 | 42/887 |
| Intracellular Transport | GO:0046907 | 6.7e−06 | 0.006 | 41/875 |
| Response to Stress | GO:0006950 | 3.6e−06 | 0.003 | 53/1230 |
| Apoptosis | GO:0006915 | 2.5e−08 | 2.5e−05 | 37/597 |
| Cell Proliferation | GO:0008283 | 1.6e−06 | 0.002 | 31/538 |

Molecular Function

| | | | | |
|---|---|---|---|---|
| Protein Binding | GO:0005515 | 3.8e−17 | 2.2e−14 | 196/4994 |
| Transcription Factor | GO:0008134 | 4.6e−09 | 2.6e−06 | 27/323 |
| Binding | | | | |
| Transcription Cofactor | GO:0003712 | 6.3e−08 | 3.6e−05 | 23/275 |
| Activity | | | | |
| DNA binding | GO:0003677 | 1.1e−07 | 6.5e−05 | 110/3020 |
| Transcription | GO:0030528 | 1.7e−08 | 9.7e−06 | 78/1816 |
| Regulator | | | | |
| Activity | | | | |
| Transcriptional | GO:0016563 | 3.1e−06 | 0.002 | 19/247 |
| Activator Activity | | | | |

Cellular Component

| | | | | |
|---|---|---|---|---|
| Nucleus | GO:0005634 | 1.0e−20 | 2.05e−18 | 214/5279 |
| Cytoplasm | GO:0005737 | 1.7e−07 | 3.4e−05 | 145/4347 |
| Endosome | GO:0005768 | 3.7e−06 | 0.001 | 10/17 |
| Golgi Stack | GO:0005795 | 1.2e−05 | 0.002 | 23/373 |

Corrected P values represent simulation based analysis for multiple hypothesis correction. The numerator for the number of annotation ratio equates to the number of genes within the parsed dataset that were determined for the statistically significant GO term, whereas the denominator represents the number of current annotations for the GO term.

Score 0 group patients had significantly elevated expression levels of retinoblastoma 1 (Rb1) and cyclin-dependent kinase inhibitor 2D (CDKN2D) with concomitant decreased expression levels of Cdc42, three Kruppel-like transcription factors (KLF7, KLF10, and KLF11), and cyclin-dependent kinase inhibitor 2B (CDKN2B; Table 4), a profile consistent with transcriptional abnormalities related to apoptosis and cell proliferation. Moreover, the apparent coordinated dysregulation of specific genes families, including the syntaxins, tubulins, RABs, and adaptor-related protein complex genes indicated that abnormalities in transcriptional regulation of cellular organization and intracellular transport did not arise by chance (Table 4).

TABLE 4

| | | |
|---|---|---|
| Gene | Fold Change | P Value |

Transcription

| | | |
|---|---|---|
| KLF7 | 2.7 | 0.02 |
| KLF10 | 9.8 | 0.03 |
| KLF11 | 3.1 | 0.01 |
| CREB1 | −2.8 | 0.02 |
| DRAP1 | 2.4 | 0.04 |
| RREB1 | 2.7 | 0.04 |
| RB1 | −2.2 | 0.04 |
| GATA5 | 8.9 | 0.05 |
| RUNX1 | 11.5 | 0.02 |
| RUNX3 | 3.5 | 0.05 |

Cell Organization and Biogenesis and
Intracellular Transport

| | | |
|---|---|---|
| CDC42 | 4.2 | 0.04 |
| MYO9B | 4.4 | 0.01 |
| RAB9A | −3.3 | 0.01 |
| RAB10 | −2.6 | 0.01 |
| AP3M2 | −2.5 | 0.02 |
| AP3S2 | 8.4 | 0.04 |
| AP4E1 | −2.8 | 0.01 |
| AP4S1 | −2.5 | 0.04 |
| STXBP2 | 2.7 | 0.03 |
| STX6 | −2.9 | 0.02 |
| STX7 | −2.8 | 0.04 |
| TUBA1 | 2.1 | 0.04 |
| H2-ALPHA | 2.4 | 0.05 |
| TUBA6 | 2.8 | 0.01 |
| TUBB4 | 5.7 | 0.02 |
| TUBB6 | 4.0 | 0.00 |

Apoptosis

| | | |
|---|---|---|
| BAG4 | −6.6 | 0.04 |
| CARD6 | −5.6 | 0.02 |
| CASP3 | −3.6 | 0.02 |
| CASP10 | −4.2 | 0.01 |
| CUL5 | −2.5 | 0.03 |
| CYCS | −2.7 | 0.05 |
| IFI16 | −5.1 | 0.02 |
| TNFSF10 | −5.4 | 0.03 |

Cell Proliferation

| | | |
|---|---|---|
| SPHK1 | 4.3 | 0.03 |
| EMP1 | 9.8 | 0.02 |
| EMP3 | 2.5 | 0.04 |
| NCK1 | −3.0 | 0.02 |
| PIM1 | 11 | 0.00 |
| SCGB3A1 | 2.3 | 0.03 |
| CDKN2D | 4.4 | 0.03 |
| CDKN2B | 2.8 | 0.03 |

*Relative fold change: Ratio of Group I (collateral score 2) to Group 2 (collateral score 0).

Significant cellular component terms, composed of the nucleus, cytoplasm, endosome, and Golgi stack, support appropriate compartmentalization of the observed biological processes and molecular functions within the S1 transcript list. A partial tabulation of differential S1 transcripts that encompass these GO terms is shown in Table 4.

To confirm that these GO terms did not arise by chance, 10 randomly permuted datasets containing the same 16 CAD patients were generated and analyzed in the same manner. The random data permutations resulted in loss of significant associative GO terms, further indicating that enriched GO terms in the S1 list represent relevant biological differences between patient groups.

Agglomerative hierarchical clustering (Eisen, et al. (1998) Proc. Natl. Acad. Sci. USA 95(25):14863-14868) and principal component analysis (PCA; Raychaudhuri, et al. (2000) Pac. Symp. Biocomput. 455-466) were used to visualize the 256 (p≦0.01) transcripts of the S2 subset. Subjects 1-8 represented patients from the score 2 coronary collateral vessel group (group 1), whereas subjects 9-16 encompassed patients from the score 0 coronary collateral vessel group (group 2). The heatmap showed strong statistical segregation in expression values between patients groups and demonstrated that the majority of the transcripts in the S2 probe set were significantly upregulated in score 0 group patients.

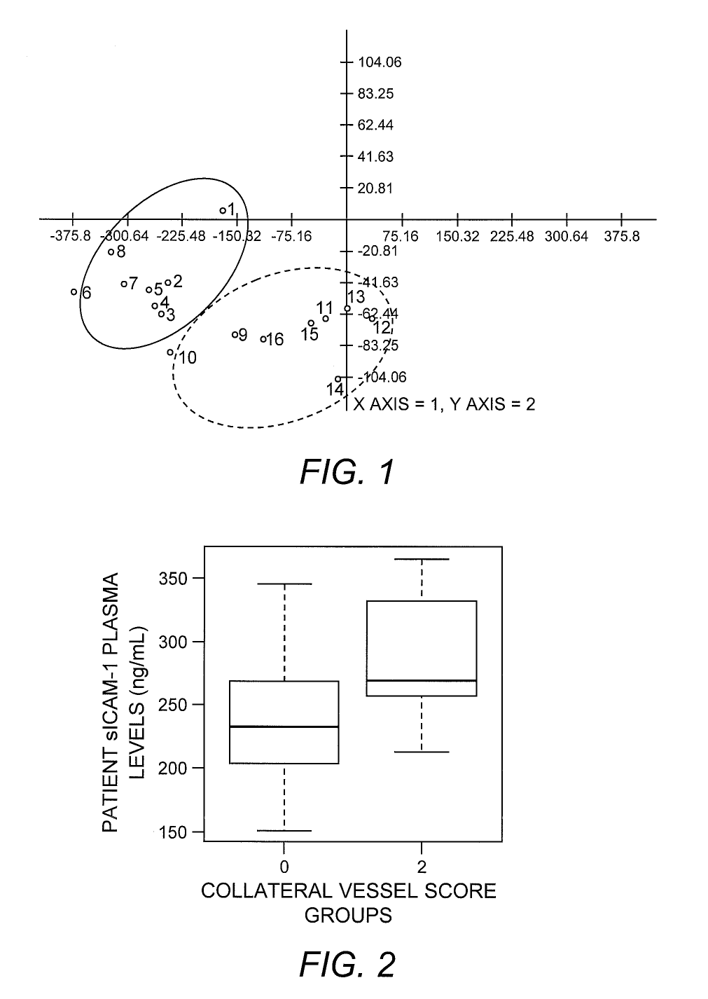

To confirm these findings, PCA was performed on the S2 transcript list. PCA analysis indicated that the first and second principal components cumulatively explained 77.96% of the variability within the 256 transcripts used for analysis (FIG. 1). Score 2 group subjects form the encircled patient cluster shown with a solid line in FIG. 1, while subjects in the score 0 shape the second patient cluster shown with a dashed line. This visualization supports the power of the S2 dataset to capture variation in expression relevant to discrimination of patient classes.

Based on expression patterns, K-Nearest Neighbors (k-NN; Theilhaber, et al. (2002) Genome Res. 12(1):165-176),

Support Vector Machines (SVM; Brown, et al. (2000) Proc. Natl. Acad. Sci. USA 97(1):262-267), C4.5 (Lim, et al. (2000) Machine Learning 40(3):203-228), and Naïve Bayes/Diagonal Linear Discriminant Analysis (DLDA; Hastie et al. (2003) The Elements of Statistical Learning: Data Mining, Inference, and Prediction. 1^{st} ed. New York: Springer-Verlag) classification algorithms were employed to assess the predictive power of this cluster of transcripts to assign patients to either the score 2 or score 0 group. Leave one out cross validation (LOOCV; Breiman (1996) Annals of Statistics 24(6):2350-2383) was used to evaluate the efficiency of each classifier (Table 5).

TABLE 5

| | | Correctly | % | Number |
|---|---|---|---|---|
| | Misclassified | Classified | Classification | of |
| Classifier | Instances | Instances | Accuracy | Genes |
| k-NN | 1 | 15 | 93.75 | 256 |
| SVM | 1 | 15 | 93.75 | 256 |
| C4.5 | 1 | 15 | 93.75 | 256 |
| DLDA | 2 | 14 | 87.50 | 256 |

Reduction of observation expression redundancy improved k-NN, SVM, and DLDA, but not C4.5 classification accuracy (Table 6).

TABLE 6

| | | | Correctly | | Number |
|---|---|---|---|---|---|
| | Feature | Misclass. | Class. | % Class. | of |
| Classifier | Selection | Instances | Instances | Accuracy | Genes |
| k-NN | X² | 0 | 16 | 100 | 3 |
| SVM | X² | 0 | 16 | 100 | 3 |
| C4.5 | X² | 1 | 15 | 93.75 | 16 |
| DLDA | X² | 0 | 16 | 100 | 4 |
| k-NN | IG | 0 | 16 | 100 | 3 |
| SVM | IG | 0 | 16 | 100 | 3 |
| C4.5 | IG | 1 | 15 | 93.75 | 21 |
| DLDA | IG | 0 | 16 | 100 | 5 |
| k-NN | RF | 0 | 16 | 100 | 8 |
| SVM | RF | 0 | 16 | 100 | 5 |
| C4.5 | RF | 2 | 14 | 87.50 | 1 |
| DLDA | RF | 0 | 16 | 100 | 8 |

After correction for expression redundancy, the improvement in patient classification accuracy was achieved, in part, by the identification the following transcripts: ARID4B, MAPKAK-2, LEPROTL1, INPP4B, GRB2-related 2. These genes represent a partial consensus of top ranked HykGene transcripts (Table 7).

TABLE 7

| | Microarray | P | qt-PCR Fold | |
|---|---|---|---|---|
| Gene | Fold Change* | Value | Change* | P Value |
| ARID4B | −4.4 | 0.001 | −4.8 | 0.007 |
| MAPKAPK-2 | 6.0 | 0.002 | 1.2 | 0.534 |
| LEPROTL1 | −5.4 | 0.001 | −3.6 | 0.001 |
| INPP4B | −7.4 | 0.007 | −9.5 | 0.008 |
| GRB2-related 2 | 4.2 | 0.008 | 2.4 | 0.031 |

*Relative fold change: Ratio of Group I (collateral score 2) to Group 2 (collateral score 0).

To confirm the HykGene findings and determine the possible effect of a cell processing bias found in the primary collateral group dataset, quantitative PCR (qPCR) analysis of differential gene expression was carried out both between these groups of CAD patients using the original patient population (Table 7), as well as in 12 additional clinically matched CAD patients all with 3 vessel disease (6 patients with collateral score 2 and 6 with collateral score 0)(Table 8). In both cases, qPCR confirmed differential abundance of the top HykGene-determined expression markers between patient classes. Furthermore, qPCR analysis of the 12 additional, 3 vessel disease patients indicated that relative collateral marker expression was not dependent upon disease severity.

TABLE 8

| | qt-PCR Fold | |
|---|---|---|
| Gene | Change* | P Value |
| ARID4B | −9.6 | 0.031 |
| MAPKAPK-2 | 1.7 | 0.485 |
| LEPROTL1 | −7.5 | 0.031 |
| INPP4B | −11 | 0.031 |
| GRB2-related 2 | 1.3 | 0.618 |

*Relative fold change: Ratio of Group I (collateral score 2) to Group 2 (collateral score 0).

To demonstrate that transcriptional changes in monocyte gene expression correlated with changes in corresponding protein expression, two additional studies were carried out using patient samples different from the original 16 patient data set. Chilibot text mining was used to determine if known relationships existed between angiogenesis, the hypothesized queried term, and statistically significant differentially expressed genes within the enriched biological process terms of the GO analysis to further narrow the list of candidates for assessment of differential protein expression. On the basis of this analysis, four proteins were identified as suitable markers of differences between the score 2 and score 0 collateral groups, namely ICAM-1, Cdc42, SSP1, and RB1. In agreement with the microarray analyses, plasma ELISA measurements of circulating soluble ICAM-1 (sICAM-1) levels in an independent group of 29 patients demonstrated significantly higher levels (287.29±12.69 vs. 235.80±12.73 ng/mL; p<0.01) in the plasma of patients with score 2 collaterals (n=14) compared to patients with score 0 collaterals (n=15) (FIG. 2).

Western blot analysis of Cdc42 expression in circulating monocytes demonstrated, in agreement with the microarray findings, more frequent expression in patients with score 2 collaterals (15 of 17 patients) vs. score 0 collaterals (6 of 17 patients, X²=10.1, p<0.01).

To further assess the possible confounding effects of disease severity on collateral group classification membership, subjects of the initial microarray analysis were then regrouped according to the angiographic extent of coronary disease to form a secondary, disease severity dataset. This dataset, which represents a reordering of patient class membership, was statistically evaluated in the same manner as the primary, collateralization dataset. Patients with 2 to 3 vessel disease (n=7) were compared to patients with 1 vessel disease (n=9). As with the primary collateral dataset, GO was used to characterize the significance of the statistically assessed disease severity dataset. Unlike the collateral dataset, evaluation of the disease severity groups indicated that the only significant molecular difference resided in protein binding.

1. A method of determining collateral artery development in a human subject with coronary artery disease comprising detecting levels of expression of markers associated with collateral artery development in a test sample from a human subject with coronary artery disease; and comparing detected levels with levels of expression of the markers in a reference sample, wherein the difference in the levels of expression is indicative of collateral artery development in the human subject.

2. The method of claim 1, wherein the markers comprise one or more of KLF7, KLF10, KLF11, CREB1, DRAP1, RREB1, RB1, GATA5, RUNX1, RUNX3, CDC42, MYO9B, RAB10, AP3M2, AP3S2, AP4E1, AP4S1, STXBP2, STX6, STX7, TUBA1, H2-ALPHA, TUBA6, TUBB4, TUBB6, BAG4, CARD6, CASP3, CASP10, CUL5, CYCS, IFI16, TNFSF10, SPHK1, EMP1, EMP3, NCK1, PIM1, SCGB3A1, CDKN2D, CDKN2B, ARID4B, MAPKAPK-2, LEPROTL1, INPP4B, GRB2-related 2, ICAM-1, or SSP1.

3. The method of claim 1, wherein the levels of nucleic acid molecules encoding the markers are detected.

4. The method of claim 1, wherein the levels of marker proteins are detected.

5. The method of claim 1, wherein the markers comprise one or more of sICAM-1, SSP1, Rb1, or Cdc42.
